(19) United States
(12) Patent Application Publication
(10) Pub. No.: US 2021/0137934 A1
(43) Pub. Date: May 13, 2021

(54) METHODS OF IDENTIFYING MYC-DRIVEN AND LIPOGENESIS-DEPENDENT NEOPLASMS AND METHODS OF TREATING THE SAME

(71) Applicant: The Board of Trustees of the Leland Stanford Junior University, Stanford, CA (US)

(72) Inventors: Arvin Gouw, San Francisco, CA (US); Dean W. Felscher, San Mateo, CA (US)

(21) Appl. No.: 16/492,500

(22) PCT Filed: Mar. 15, 2018
(86) PCT No.: PCT/US2018/022733

§ 371 Date: Sep. 9, 2019

Related U.S. Application Data
(60) Provisional application No. 62/472,510, filed on Mar. 16, 2017.

(51) Int. Cl. A61K 31/5377; A61K 31/34; A61K 31/365; A61K 31/475; A61K 31/664

(52) U.S. Cl. CPC A61K 31/5377; A61K 31/34; A61K 31/664; A61K 31/475; A61K 31/365

(57) ABSTRACT

Provided are methods of identifying MYC-Driven and/or lipogenesis-dependent neoplasms. Also provided are methods of treating the MYC-Driven neoplasms and methods of treating lipogenesis-dependent neoplasms. Methods of identifying therapeutic agents that are effective against MYC-driven neoplasms are also provided.

### CROSS REFERENCE

This application claims benefit of U.S. Provisional Patent Application No. 62/472,510, filed Mar. 16, 2017 which application is incorporated herein by reference in its entirety.

### GOVERNMENT RIGHTS

This invention was made with Government support under contract CA184384 awarded by the National Institutes of Health. The Government has certain rights in the invention.

### BACKGROUND

The MYC oncogene is often genetically activated and/or overexpressed in human cancer. MYC is a transcription factor that dimerizes with MAX to bind DNA and amplifies gene expression genome-wide to regulate many cellular programs including cellular proliferation, growth, metabolism, differentiation, survival, self-renewal, angiogenesis, and immune evasion. MYC inactivation can result in tumor regression.

MYC regulates genes involved in glycolysis and glutaminolysis, which are required for energy production and macromolecular synthesis. However, it has not been studied if MYC regulates lipogenesis.

Fatty acid synthesis is required to generate complex lipids like cholesterol and glycerophospholipids. The fatty acid synthesis pathway includes multiple steps. First, citrate produced from the TCA cycle is released into the cytoplasm and converted into acetyl-CoA by ATP citrate lyase (ACLY). Acetyl-CoA can also be derived from acetate through the activity of acetyl-CoA synthetases (ACSs). The de novo fatty acid synthesis starts with the production of malonyl-CoA from acetyl-CoA by ACACA. Malonyl-CoA is then converted into palmitate FA(16:0) by Fatty Acid Synthase (FASN) that sequentially elongates the fatty acid chain. Palmitate is then converted to stearate FA(18:0), that in turn can be monounsaturated to oleate FA(18:1) by stearoyl-CoA desaturase (SCD).

The pathway critical to lipogenesis is regulated through the Sterol Regulatory Element Binding Proteins (SREBPs). SREBPs are transcription factors for both fatty acid synthesis and cholesterol biosynthesis that consist of two splice variants, SREBP-1a and SREBP-1c, and SREBP-2. SREBPs are synthesized as precursors that are anchored in the endoplasmic reticulum (ER) via two transmembrane helices in association with an ER retention protein, termed Insig. Upon activation by diminished cholesterol levels, the SREBP cleavage activating protein (SCAP) dissociates SREBP from Insig. The liberated N-terminal cytosolic SREBP then translocates into the nucleus and activates genes involved in lipid metabolism. SREBP1 activates genes involved in fatty acid synthesis, such as ACLY, ACS, ACACA, FASN, and SCD. SREBP2 activates genes involved in mevalonate and cholesterol synthesis.

### SUMMARY

Provided are methods of identifying MYC-Driven and/or lipogenesis-dependent neoplasms. Also provided are methods of treating the MYC-Driven neoplasms and methods of treating lipogenesis-dependent neoplasms. Methods of identifying therapeutic agents that are effective against MYC-driven neoplasms are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1a-1c: Upregulation of lipid metabolic genes by MYC. 1a) diagram of the analyzed lipid metabolic pathways and their upregulation by MYC. 1b) RNA-seq shows MYC increases expression of lipogenesis genes comparing MYC off and 24 hours MYC on p493-6 cells. 1c)) qPCR show upregulation of fatty acid synthesis genes in following 24 hours of MYC inactivation on T-ALL (4188), BCL (P493), HCC (EC4), and RCC (E28).

FIG. 2a-2d: MYC orchestrates glycolysis, glutaminolysis, and lipogenesis. 2a) MYC directly binds to the gene promoters of glycolysis, glutaminolysis, and lipogenesis to induce transcription. 2b) MYC binds in a time-dependent manner upon MYC activation in P493 cells. 2c) Nuclear Run-on analysis shows enrichment of lipogenesis gene transcription over a time course following MYC activation in p493 cells. 2d) Timecourse binding profile of Myc in P493 cells from publically available database. UCSC Genome Browser display of publicly available MYC ChIP-Seq in p493-6 cells 0, 1 and 24 hours after tetracycline release. Y-axis shows 1 to 12 reads per million.

FIG. 3a-3b: MYC regulates SREBP and together regulates lipogenesis. 3b) siRNA knockdown of control or SREBP1 under MYC ON and OFF conditions in mouse HCC EC4 line shows that the presence of both MYC and SREBP1 is required for the induction of the fatty acid synthesis genes: ACLY, ACACA, FASN, and SCD1.

FIG. 4a-4d: Glucose as the predominant source of lipogenesis by MYC. 4a) Scheme of glucose and glutamine consumption for de novo lipogenesis. 4b) DESI-MSI showing induction of oleate by MYC in our RCC, HCC, BCL, and LCC models. 4c) glucose contribution to lipogenesis by mass spectrometry in Myc ON P493-6 cells. 4d) glucose and glutamine contribution to lipogenesis via NMR in MYC ON vs OFF P493-6 cells. For close-up look see fig. S3a.

FIG. 5a-5b: Lipid signature of MYC-induced HCC, RCC, T-ALL, LCC bronchiolar (b), and adenoma (a). Gray color represents undetected ion signal. 5a) PGs are upregulated while PIs are downregulated by MYC compared to normal tissue for HCC, RCC, T-ALL, LCC (b), and LCC (a). 5b) PGs downregulated while PIs are upregulated upon MYC inactivation in HCC, RCC, and T-ALL.

FIG. 6a-6b: Dynamic changes of phospholipid profile in RCC by DESI-MSI. 6a) histology of examined kidney sections shows representative decrease of short PGs and increase of long PGs. 6b) representative mass spectra of various levels of MYC in RCC compared to control kidney tissue.

FIG. 7a-7c: MYC upregulates PG synthesis and elongation genes in RCC. 7a) MYC elongates desaturated fatty acids, increases the fatty acid pool, and further upregulates PG synthesis genes. MYC RCC upon MYC activation (weeks 0-4) and inactivation (days 1-10) shows upregulation of 7b.) PG synthesis gene expression and 7c.) fatty acid chain elongases.

FIG. 8a-8c: TOFA prevents RCC tumorigenesis and progression in vivo. 8a) TOFA significantly reduces kidney tumor progression upon treatment at week 2, and completely prevents tumorigenesis upon initial treatment from the beginning at week 0. 8b) TOFA regresses human RCC 786-O xenograft growth in NSG mice. 8c) RCC and kidney H&E sections.

FIG. 9a-9b: TOFA treatment and prevention of RCC 9a) H&E of examined mouse kidney sections and overview of the time-course experiments. 9b) tumor inhibition in human 786-O cell line by TOFA showing representative PI distribution and long PG distribution upon TOFA treatment of 15 day MYC on tumor and prevention started at day 0 MYC on.

FIG. 10: MYC as master regulator of lipid metabolic pathways: fatty acid synthesis, fatty acid elongation, and finally PG synthesis.

FIG. 11 provides FIG. S1a-S1e: MYC upregulates lipid metabolic genes. S1a) Quantification (reads per million) of publicly available MYC ChIP-Seq in p493-6 cells 0, 1 and 24 hours after tetracycline release. S1b) Binding profile of MYC on fatty acid synthesis genes from publicly available MYC ChIP-Seq in p493-6 cells 0, 1 and 24 hours after tetracycline release. S1c) Quantification (reads per million) of publically available MYC ChIP-Seq on cholesterol biosynthesis genes. S1d) RNA-seq shows MYC increases expression of cholesterol biosynthesis genes comparing MYC off and 24 hours MYC on p493-6 cells. S1e) Quantification of binding data of MYC on fatty acid synthesis genes of publicly available MYC ChIP-Seq in p493-6 cells 0, 1 and 24 hours after tetracycline release.

FIG. 12 provides FIG. S2a-S2d: Timecourse qPCR and binding data of publicly available MYC ChIP-Seq in p493-6 cells 0, 1 and 24 hours after tetracycline release. S2a) MYC and MAX binding on cholesterol biosynthesis genes based on ChIP-seq data from publically available data. Timecourse mRNA levels in p493 cells 0, 1, and 24 hours after tetracycline release of S2b) glycolysis genes, S2c) glutaminolysis genes, and S2d) fatty acid synthesis genes.

FIG. 13 provides FIG. S3a-S3c: Lipogenesis and fatty acid oxidation. S3a) close-up look into glucose and glutamine contribution to lipogenesis via NMR in Myc ON vs OFF P493-6 cells S3b) upon MYC inactivation 13C-labeled oleate reveals that oleate accumulated in MYC-OFF cells. S3c) meanwhile upon MYC activation 13C-label glucose is converted to fatty acids.

FIG. 14 provides FIG. S4a-S4e: Dynamic changes of phospholipid profile in RCC by DESI-MSI. S4a) Quantification of representative distribution of oleate across four MYC-inducible systems. S4b) Representative DESI-MSI distribution of short PGs, and S4c) long PGs. S4d) Short PGs decrease upon MYC activation, which then increases upon MYC inactivation. S4e) Long PGs increase upon MYC activation, which in turn decrease upon MYC inactivation.

FIG. 15 provides FIG. S5a-S5b: Dynamic changes of phospholipid profile in RCC by DESI-MSI. S5a) Representative DESI-MSI distribution of PIs. Molecular structures of these phospholipids are given in the left panel. S5b) PIs decrease upon MYC activation, and increase with MYC inactivation.

FIG. 16 provides FIG. S6a-S6b: Binding data of publicly available MYC ChIP-Seq in p493-6 cells 0, 1 and 24 hours after tetracycline release on the promoters of CDP-DAG synthesis genes (S6a), and elongases (S6b).

FIG. 17 provides FIG. S7a-S7e: Inhibition of lipogenesis. S7a) scheme of possible lipogenesis inhibition: Dose-dependent inhibition of lipogenesis in P493-6 lymphoma by Cerulenin, an FASN inhibitor and TOFA, an ACACA inhibitor. P493 cell proliferation is decreased in a dose-dependent manner following administration of Cerulenin (S7b) and TOFA (S7c). TOFA inhibitory action in E28 RCC cells are similar (S7d) which can be partially rescued by oleate (S7e).

FIG. 18 provides FIG. S8a-S8d: TOFA treatment and prevention of RCC. Representative distribution of PGs (S6a), and PIs (S6b) upon TOFA treatment of 15 day MYC on tumor and prevention at day 0 MYC on. Representative distribution of PGs (S6c) and PIs (S6d) upon TOFA treatment of 15 day human RCC 786-0 subcutaneous xenografts in NSG mice.

FIG. 19 provides Table 1.

FIG. 20 provides Table 2: Selected glycerophospholipid ion abundance changes relative to control tissue across various MYC-induced tumors—PGs.

FIG. 21 provides Table 3: Selected glycerophospholipid ion abundance changes relative to control tissue across various MYC-induced tumors—PIs.

FIG. 22 provides Table 4: Selected glycerophospholipid ion abundance changes relative to MYC OFF across various MYC-induced tumors—PGs.

FIG. 23 provides Table 5: Selected glycerophospholipid ion abundance changes relative to MYC OFF across various MYC-induced tumors—PIs.

### DEFINITIONS

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁵ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “antibody” and “immunoglobulin”, as used herein, are used interchangeably may generally refer to whole or intact molecules or fragments thereof and modified and/or conjugated antibodies or fragments thereof that have been modified and/or conjugated. The immunoglobulins can be divided into five different classes, based on differences in the amino acid sequences in the constant region of the heavy chains. All immunoglobulins within a given class will have very similar heavy chain constant regions. These differences can be detected by sequence studies or more commonly by serological means (i.e. by the use of antibodies directed to these differences). Immunoglobulin classes include IgG (Gamma heavy chains), IgM (Mu heavy chains), IgA (Alpha heavy chains), IgD (Delta heavy chains), and IgE (Epsilon heavy chains).

Antibody or immunoglobulin may refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated as V_{H}) and a heavy chain constant region (abbreviated as C_{H}). The heavy chain constant region typically is comprised of three domains, C_{H}1, C_{H}2, and C_{H}3. Each light chain typically is comprised of a light chain variable region (abbreviated as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Whole or largely intact antibodies are generally multivalent, meaning they may simultaneously bind more than one molecule of antigen whereas antibody fragments may be monovalent. Antibodies produced by an organism as part of an immune response are generally monospecific, meaning they generally bind a single species of antigen. Multivalent monospecific antibodies, i.e. antibodies that bind more than one molecule of a single species of antigen, may bind a single antigen epitope (e.g., a monoclonal antibody) or multiple different antigen epitopes (e.g., a polyclonal antibody).

Multispecific (e.g., bispecific) antibodies, which bind multiple species of antigen, may be readily engineered by those of ordinary skill in the art and, thus, may be encompassed within the use of the term “antibody” used herein where appropriate. Also, multivalent antibody fragments may be engineered, e.g., by the linking of two monovalent antibody fragments. As such, bivalent and/or multivalent antibody fragments may be encompassed within the use of the term “antibody”, where appropriate, as the ordinary skilled artisan will be readily aware of antibody fragments, e.g., those described below, which may be linked in any convenient and appropriate combination to generate multivalent monospecific or polyspecific (e.g., bispecific) antibody fragments.

Antibody fragments include but are not limited to antigen-binding fragments (Fab or F(ab), including Fab′ or F(ab′), (Fab)₂, F(ab′)₂, etc.), single chain variable fragments (scFv or Fv), “third generation” (3G) molecules, etc. which are capable of binding the epitopic determinant. These antibody fragments retain some ability to selectively bind to the subject antigen, examples of which include, but are not limited to:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab)₂, the fragment of the antibody that can be obtained by treating whole antibody with the enzyme pepsin without subsequent reduction;

(4) F(ab)₂ is a dimer of two Fab′ fragments held together by two disulfide bonds;

(5) Fv, defined as a genetically engineered fragment containing the variable region of the light chain and the variable region of the heavy chain expressed as two chains;

(6) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, tetrabodies, etc. which may or may not be polyspecific (see, for example, WO 94/07921 and WO 98/44001) and

(7) “3G”, including single domain (typically a variable heavy domain devoid of a light chain) and “miniaturized” antibody molecules (typically a full-sized Ab or mAb in which non-essential domains have been removed).

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with cancer; those suspected of having cancer; etc.).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent that inhibits a target gene (e.g., a MYC-dependent target gene, and the like) and/or compositions is an amount that is sufficient, when administered to the individual, to palliate, ameliorate, stabilize, reverse, prevent, slow or delay the progression of the disease state (e.g., cancer, etc.) by, for example, inhibiting the growth of, inducing death of or otherwise preventing the clinical progressing of a MYC-dependent cancer present in the subject.

### DETAILED DESCRIPTION

Provided are methods of identifying MYC-Driven and/or lipogenesis-dependent neoplasms. Also provided are methods of treating the MYC-Driven neoplasms and methods of treating lipogenesis-dependent neoplasms. Methods of identifying therapeutic agents that are effective against MYC-driven neoplasms are also provided.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed

### Methods

As summarized above, the present disclosure provides methods of identifying MYC-Driven and/or lipogenesis-dependent neoplasms; methods of treating the MYC-Driven neoplasms; methods of treating lipogenesis-dependent neoplasms; and the like.

Methods of treating a subject for a MYC-driven neoplasm may include administering to the subject an effective amount of a lipogenesis inhibitor to treat the subject for the MYC-driven neoplasm. Methods of treating a subject for a lipogenesis-dependent neoplasm may include comparing a lipogenesis profile obtained from a subject having a neoplasm with a reference lipogenesis profile to classify whether the neoplasm is lipogenesis-dependent and administering to the subject an effective amount of a lipogenesis inhibitor, when the neoplasm is classified as lipogenesis-dependent, to treat the subject for the lipogenesis-dependent neoplasm.

The provided methods may find use with subjects having a variety of different neoplasms, including but not limited to cancers. Relevant cancers include tumors (e.g., solid tumors (e.g., sarcomas and carcinomas) and blood cancers. Non-limited examples of various cancers to which the subject methods may be applied include: Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

In some instances, a subject to which the provided methods may be applied may be a subject having a hematological (i.e., blood) cancer, e.g., a leukemia or a lymphoma. Non-limiting examples of hematological cancers include: Acute Lymphoblastic Leukemia, Adult; Acute Lymphoblastic Leukemia, Childhood; Acute Myeloid Leukemia, Adult; Acute Myeloid Leukemia, Childhood; Chronic Lymphocytic Leukemia; Chronic Myelogenous Leukemia; Hairy Cell Leukemia; AIDS-Related Lymphoma; Cutaneous T-Cell Lymphoma (see Mycosis Fungoides and the Sézary Syndrome); Hodgkin Lymphoma, Adult; Hodgkin Lymphoma, Childhood; Hodgkin Lymphoma During Pregnancy; Mycosis Fungoides; Non-Hodgkin Lymphoma, Adult; Non-Hodgkin Lymphoma, Childhood; Non-Hodgkin Lymphoma During Pregnancy; Primary Central Nervous System Lymphoma; Sezary Syndrome; T-Cell Lymphoma, Cutaneous (see Mycosis Fungoides and the Sezary Syndrome); Waldenström Macroglobulinemia (see Non-Hodgkin Lymphoma) Chronic Myeloproliferative Neoplasms; Langerhans Cell Histiocytosis; Multiple Myeloma/Plasma Cell Neoplasm; Myelodysplastic Syndromes; Myelodysplastic/Myeloproliferative Neoplasms; and the like.

In some instances, a subject to which the provided methods may be applied may be a subject having a carcinoma (e.g., an adenocarcinoma or a squamous cell carcinoma). Non-limiting examples of carcinomas include: acinar carcinoma, acinic cell carcinoma, acinous carcinoma, adenocystic carcinoma, adenoid cystic carcinoma, adenosquamous carcinoma, adnexal carcinoma, adrenocortical carcinoma, alveolar carcinoma, ameloblastic carcinoma, apocrine carcinoma, basal cell carcinoma, bronchioloalveolar carcinoma, bronchogenic carcinoma, cholangiocellular carcinoma, chorionic carcinoma, clear cell carcinoma, colloid carcinoma, colorectral carcinoma, cribriform carcinoma, ductal carcinoma in situ, embryonal carcinoma, carcinoma en cuirasse, endometrioid carcinoma, epidermoid carcinoma, carcinoma ex mixed tumor, carcinoma ex pleomorphic adenoma, follicular carcinoma of thyroid gland, hepatocellular carcinoma, carcinoma in si'tu, intraductal carcinoma, Hürthle cell carcinoma, inflammatory carcinoma of the breast, large cell carcinoma, invasive lobular carcinoma, lobular carcinoma, lobular carcinoma in situ (LCIS), medullary carcinoma, meningeal carcinoma, Merkel cell carcinoma, mucinous carcinoma, mucoepidermoid carcinoma, nasopharyngeal carcinoma, non-small cell carcinoma, non-small cell lung carcinoma (NSCLC), oat cell carcinoma, papillary carcinoma, renal cell carcinoma, scirrhous carcinoma, sebaceous carcinoma, carcinoma sim'plex, signet-ring cell carcinoma, small cell carcinoma, small cell lung carcinoma, spindle cell carcinoma, squamous cell carcinoma, terminal duct carcinoma, transitional cell carcinoma, tubular carcinoma, verrucous carcinoma, and the like.

Methods of the present disclosure may find use in analyzing and/or treating various cancers including but not limited to e.g., liver cancers, kidney cancers, blood cancer (e.g., lymphoma), lung cancers, etc. In some instances, the subject methods find use in analyzing and/or treating MYC-induced renal cell carcinoma (RCC). In some instances, the subject methods find use in analyzing and/or treating MYC-induced T-cell lymphoma (T-ALL). In some instances, the subject methods find use in analyzing and/or treating MYC-induced lung cell carcinoma. In some instances, the subject methods find use in analyzing and/or treating MYC-induced hepatocellular carcinoma (HCC). In some instances, the subject cancers may be MYC driven cancers. In some instances, the cancers may be cancers in which MYC induces fatty acid synthesis and/or the expression of fatty acid synthesis genes.

Aspects of the present methods, although described in reference to RCC below, may be generally applicable to various neoplasms, including but not limited to e.g., those described herein. Aspects of the present methods include treating a subject for a MYC-driven renal cell carcinoma (RCC) by administering to the subject an effective amount of a lipogenesis inhibitor to treat the subject for the MYC-driven RCC. In some instances, the subject is identified as having a MYC-driven RCC. In some instances, the administering results in regression of the MYC-driven RCC.

In some aspects, methods of the present include the use of a lipogenesis profile. By “lipogenesis profile” is meant a representation of the lipids or a subset thereof present in a cellular sample (e.g., a cell, a population of cells, a tissue, an organ, ect.) which may or may include quantification of the absolute or relative amounts of the subject lipids or subset thereof. In some instances, a lipid profile or a lipogenesis profile may be obtained for glycerophospholipids or a subset thereof. Lipogenesis profiles may be compared. In some instances, a lipogenesis profile may be compared to a control (e.g., a normal tissue, a MYC “ON” control, a MYC “OFF” control, or the like). In some instances, a lipogenesis profile may be compared to a reference lipogenesis profile, e.g., a reference lipogenesis profile obtained from a control (e.g., a normal tissue reference lipogenesis profile, a MYC “ON” control reference lipogenesis profile, a MYC “OFF” control reference lipogenesis profile, or the like). In some instances, a lipogenesis profile may be employed to assign a particular lipogenesis state to a cellular sample (e.g., a cellular sample of a neoplasm, such as an RCC). Assigning a particular lipogenesis state to a cellular sample may include classifying the cellular sample as lipogenesis-dependent, e.g., when the lipogenesis profile obtained includes increased glycerophosphoglycerols as compared to a reference lipogenesis profile or decreased glycerophosphoinositols compared to the reference lipogenesis profile.

Any convenient method may be employed for obtaining a lipogenesis profile of the present methods. In some aspects, the methods of the present disclosure may include lipogenesis profiles obtained using mass spectrometry (MS) and as such may be mass spectrometry (MS) lipogenesis profiles. Any convenient and appropriate MS technology may be employed, including but not limited to e.g., desorption electrospray ionization mass spectrometry imaging (DESI-MSI).

In some instances, methods of treating a subject may include administering to the subject an effective amount of a lipogenesis inhibitor, when the neoplasm of the subject is classified as lipogenesis-dependent, to treat the subject for the lipogenesis-dependent neoplasm.

In some instances, the methods of the present disclosure include treating a subject for a neoplasm by administering the subject an effective amount of one or more inhibitors of ATP citrate lyase (ACLY). ATP citrate lyase is the primary enzyme responsible for the synthesis of cytosolic acetyl-CoA in many tissues. The enzyme is a tetramer (relative molecular weight approximately 440,000) of apparently identical subunits. It catalyzes the formation of acetyl-CoA and oxaloacetate from citrate and CoA with a concomitant hydrolysis of ATP to ADP and phosphate. The product, acetyl-CoA, serves several important biosynthetic pathways, including lipogenesis and cholesterogenesis. In nervous tissue, ATP citrate-lyase may be involved in the biosynthesis of acetylcholine. Multiple transcript variants encoding distinct isoforms have been identified for this gene.

Any useful inhibitor of the ACLY target gene and/or encoded product thereof may be employed in the subject methods. Non-limiting examples of useful inhibitors include but are not limited to e.g., non-peptide small molecule antagonists, peptide antagonists, interfering RNAs (e.g., siRNA, shRNA, etc.), antibodies (e.g., neutralizing antibodies, function blocking antibodies, etc.), aptamers, and the like. In some instances, the effectiveness of an inhibitor may be confirmed using an in vitro or in vivo assay, including e.g., where the effectiveness of the inhibitor is compared to an appropriate control or standard, e.g., a conventional therapy for the condition, etc.

Non-limiting examples of ACLY inhibitors include but are not limited to e.g., anti-ACLY antibodies, ACLY inhibitory nucleic acids, small molecule ACLY antagonists, and the like. Non-limiting examples of small molecule ACLY antagonists include but are not limited to e.g., 3,5-Dichloro-2-hydroxy-N-(4-methoxy[1,1′-biphenyl]-3-yl)-benzenesulfonamide (BMS 303141); 3,3,14,14-Tetramethylhexadecanedioic acid (MEDICA 16); (3R,5S)-rel-5-[6-(2,4-Dichlorophenyl)hexyl]tetrahydro-3-hydroxy-2-oxo-3-furanacetic acid (SB 204990); 8-Hydroxy-2,2,14,14-tetramethylpentadecanedioic acid (ETC-1002); and the like.

In some instances, the methods of the present disclosure include treating a subject for a neoplasm by administering the subject an effective amount of one or more inhibitors of acetyl-CoA carboxylase alpha (ACACA). ACACA is a complex multifunctional enzyme system. ACC is a biotin-containing enzyme which catalyzes the carboxylation of acetyl-CoA to malonyl-CoA, the rate-limiting step in fatty acid synthesis. There are two ACC forms, alpha and beta, encoded by two different genes. ACC-alpha is highly enriched in lipogenic tissues. The enzyme is under long term control at the transcriptional and translational levels and under short term regulation by the phosphorylation/dephosphorylation of targeted serine residues and by allosteric transformation by citrate or palmitoyl-CoA. Multiple alternatively spliced transcript variants divergent in the 5′ sequence and encoding distinct isoforms have been found for this gene.

Any useful inhibitor of the ACACA target gene and/or encoded product thereof may be employed in the subject methods. Non-limiting examples of useful inhibitors include but are not limited to e.g., non-peptide small molecule antagonists, peptide antagonists, interfering RNAs (e.g., siRNA, shRNA, etc.), antibodies (e.g., neutralizing antibodies, function blocking antibodies, etc.), aptamers, and the like. In some instances, the effectiveness of an inhibitor may be confirmed using an in vitro or in vivo assay, including e.g., where the effectiveness of the inhibitor is compared to an appropriate control or standard, e.g., a conventional therapy for the condition, etc.

Non-limiting examples of ACACA inhibitors include but are not limited to e.g., anti-ACACA antibodies, ACACA inhibitory nucleic acids, small molecule ACACA antagonists, and the like. Non-limiting examples of small molecule ACACA antagonists include but are not limited to e.g., 5-(tetradecyloxy)-2-furancarboxylic acid (TOFA); [(3R)-1′-(9-anthracenylcarbonyl)[1,4′-bipiperidin]-3-yl]-4-morpholinyl-methanone (CP 640186); 1,4-Dihydro-1′-[2-methyl-1H-benzimidazol-6-yhcarbonyl]-1-(1-methylethyl)-spiro[5H-indazole-5,4′-piperidin]-7(6H)-one (PF 05175157); 2′-(tert-Butyl)-1-(2-methoxyquinoline-7-carbonyl)-4′,6′-dihydrospiro[piperidine-4,5′-pyrazolo[3,4-c]pyridin]-7′(2′H)-one; 1,17-Dihydroxy-10,11,18-trimethoxy-2,14,16-trimethyl-5-phenyl-4,19-dioxabicyclo[13.3.1]nonadec-12-en-3-one (Soraphen A); ND-630; ND-654; (S)-(+)-4-[1-(4-tert-Butylphenyl)-2-oxo-pyrrolidin-4-yl]methoxybenzoic acid (S-2E); 8-hydroxy-2,2,14,14-tetramethylpentadecanediotic acid (ESP-55016); and the like. Non-limiting examples of ACACA inhibitors may also include those agents described in Corbett et al., Bioorg Med Chem Lett. 2010; 20(7):2383-8; Harrimana et al. Proc Natl Acad Sci USA. 2016; 113(13):E1796-805 and Bourbeau & Bartberger J Med Chem. 2015; 58(2):525-36; the disclosures of which are incorporated herein by reference in their entirety.

In some instances, the methods of the present disclosure include treating a subject for a neoplasm by administering the subject an effective amount of one or more inhibitors of fatty acid synthase (FASN). The enzyme encoded by the FASN gene is a multifunctional protein. Its main function is to catalyze the synthesis of palmitate from acetyl-CoA and malonyl-CoA, in the presence of NADPH, into long-chain saturated fatty acids.

Any useful inhibitor of the FASN target gene and/or encoded product thereof may be employed in the subject methods. Non-limiting examples of useful inhibitors include but are not limited to e.g., non-peptide small molecule antagonists, peptide antagonists, interfering RNAs (e.g., siRNA, shRNA, etc.), antibodies (e.g., neutralizing antibodies, function blocking antibodies, etc.), aptamers, and the like. In some instances, the effectiveness of an inhibitor may be confirmed using an in vitro or in vivo assay, including e.g., where the effectiveness of the inhibitor is compared to an appropriate control or standard, e.g., a conventional therapy for the condition, etc.

Non-limiting examples of FASN inhibitors include but are not limited to e.g., anti-FASN antibodies, FASN inhibitory nucleic acids, small molecule FASN antagonists, and the like. Non-limiting examples of small molecule FASN antagonists include but are not limited to e.g., Cerulenin; N-Formyl-L-leucine (1S)-1-[[(2S,3S)-3-hexyl-4-oxo-2-oxetanyl]methyl]dodecyl ester (Orlistat); (2R*,3S*)-Tetrahydro-4-methylene-2-octyl-5-oxo-3-furancarboxylic acid (C-75); 3-(3,4,5-Trihydroxybenzoyloxy)naphthalen-1-yl 3,4,5-trihydroxybenzoate (G-28UCM); 4-[4-(5-Benzofuranyl)phenyl]-5-[[(3S)-1-(cyclopropylcarbonyl)-3-pyrrolidinyl]methyl]-2,4-dihydro-3H-1,2,4-triazol-3-one (GSK 2194069); and the like.

In some instances, the methods of the present disclosure include treating a subject for a neoplasm by administering the subject an effective amount of one or more inhibitors of stearoyl-CoA desaturase (SCD). The SCD gene encodes an enzyme involved in fatty acid biosynthesis, primarily the synthesis of oleic acid. The protein belongs to the fatty acid desaturase family and is an integral membrane protein located in the endoplasmic reticulum. Transcripts of approximately 3.9 and 5.2 kb, differing only by alternative polyadenlyation signals, have been detected. A gene encoding a similar enzyme is located on chromosome 4 and a pseudogene of this gene is located on chromosome 17.

Any useful inhibitor of the SCD target gene and/or encoded product thereof may be employed in the subject methods. Non-limiting examples of useful inhibitors include but are not limited to e.g., non-peptide small molecule antagonists, peptide antagonists, interfering RNAs (e.g., siRNA, shRNA, etc.), antibodies (e.g., neutralizing antibodies, function blocking antibodies, etc.), aptamers, and the like. In some instances, the effectiveness of an inhibitor may be confirmed using an in vitro or in vivo assay, including e.g., where the effectiveness of the inhibitor is compared to an appropriate control or standard, e.g., a conventional therapy for the condition, etc.

Non-limiting examples of SCD inhibitors include but are not limited to e.g., anti-SCD antibodies, SCD inhibitory nucleic acids, small molecule SCD antagonists, and the like. Non-limiting examples of small molecule SCD antagonists include but are not limited to e.g., 4-(2-Chlorophenoxy)-N-[3-[(methylamino)carbonyl]phenyl]-1-piperidinecarboxamide (A 939572); 4-Pyridinecarboxylic acid 2-phenylhydrazide (PluriSln 1); 2-[5-[3-[4-(2-bromo-5-fluorophenoxy)piperidin-1-yl]-1,2-oxazol-5-yl]tetrazol-2-yl]acetic acid (MK-8245); 2-methyl-5-(6-(4-(2-(trifluoromethyl)phenoxy)piperidin-1-yl)pyridazin-3-yl)-1,3,4-thiadiazole (MF-438); 3-[4-(2-Chloro-5-fluorophenoxy)-1-piperidinyl]-6-(5-methyl-1,3,4-oxadiazol-2-yl)pyridazine (CAY 10566); N-(2-(6-(3,4-dichlorobenzylamino)-2-(4-methoxyphenyl)-3-oxopyrido[2,3-b]pyrazin-4(3H)-yl)ethyl) acetamide (CVT-11127); MF-152; LCF369; CVT-11,563; CVT-12,012; DSR-4029; GSK993; HYR-061; and the like.

In some instances, methods of the present disclosure may include administering to a subject a lipogenesis inhibitor, including e.g., where two or more lipogenesis inhibitors are administered including e.g., 3 or more, 4 or more, 5 or more, etc.

An individual to be treated according to the present methods will generally be an individual with a neoplasia. As used herein “neoplasia” includes any form of abnormal new tissue formation; and the like. In some cases, the individual has recently undergone treatment for neoplasia (e.g., cancer, a tumor, etc.) and are therefore at risk for recurrence. In some instances, the individual has not recently or previously undergone treatment for a neoplasia (e.g., cancer, a tumor, etc.) but has been newly diagnosed with a neoplasia. Any and all neoplasia are suitable neoplasia to be treated by the subject methods e.g., utilizing an agent described herein or a herein described treatment kit.

Pharmaceutical Compositions

The compositions (e.g., those including one or more inhibitor of lipogenesis) of this disclosure can be supplied in the form of a pharmaceutical composition. Any suitable pharmaceutical composition may be employed, described in more detail below. As such, in some instances, methods of the present disclosure may include administering an inhibitor in a composition comprising an excipient (e.g., an isotonic excipient) prepared under sufficiently sterile conditions for administration to a mammal, e.g., a human.

Administration of an inhibitor to a subject, as described herein, may be performed employing various routes of administration. The route of administration may be selected according to a variety of factors including, but not necessarily limited to, the condition to be treated, the formulation and/or device used, the patient to be treated, and the like. Routes of administration useful in the disclosed methods include but are not limited to oral and parenteral routes, such as intravenous (iv), intraperitoneal (ip), rectal, topical, ophthalmic, nasal, and transdermal. Formulations for these dosage forms are described herein.

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (host) being treated.

Therapeutically effective doses of a subject compound or pharmaceutical composition can be determined by one of skill in the art, with a goal of achieving local (e.g., tissue) concentrations that are at least as high as the IC50 of an applicable compound disclosed herein.

The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, Md. 20857, the disclosure of which is incorporated herein by reference).

Conversion of Animal Doses to Human Equivalent
Doses Based on Body Surface Area

| | To Convert Animal | To Convert Animal Dose in mg/kg | |
|---|---|---|---|
| | Dose in mg/kg to | to HED^{a} in mg/kg, Either: | |
| | Dose in mg/m², | Divide Animal | Multiply Animal |
| Species | Multiply by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| monkey | | | |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)0.33.
^{b}This km value is provided for reference only since healthy children will rarely be volunteers for phase 1 trials.
^{c}For example, cynomolgus, rhesus, and stumptail.

A pharmaceutical composition comprising a subject compound (i.e., an inhibitory agent or a combination thereof) may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, tablet, pill, pellet, capsule, powder, syrup, elixir or any other dosage form suitable for administration.

A subject compound may be administered to the host using any convenient means capable of resulting in the desired reduction in disease condition or symptom. Thus, a subject compound can be incorporated into a variety of formulations for therapeutic administration. More particularly, a subject compound can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the subject compounds can be formulated for use in human or veterinary medicine. Particular formulations of a disclosed pharmaceutical composition may depend, for example, on the mode of administration and/or on the location of the infection to be treated. In some embodiments, formulations include a pharmaceutically acceptable carrier in addition to at least one active ingredient, such as a subject compound. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A subject compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A subject compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

In some embodiments, a subject compound can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

A subject compound can be utilized in aerosol formulation to be administered via inhalation. A subject compound can be formulated into pressurized acceptable propellants such as dichlorodifluoromethane, propane, nitrogen and the like.

Furthermore, a subject compound can be made into suppositories by mixing with a variety of bases such as emulsifying bases or water-soluble bases. A subject compound can be administered rectally via a suppository. The suppository can include vehicles such as cocoa butter, carbowaxes and polyethylene glycols, which melt at body temperature, yet are solidified at room temperature.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions comprising a subject compound may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

Screening Methods

As summarized above, methods are also provided for identifying therapeutic agents that are effective against MYC-driven neoplasms, including but not limited to e.g., MYC-driven RCC. Such methods may be generally referred to as methods of screening. Methods are also provided which include treating a subject, as described above, through administering to the subject a therapeutic agent identified using such screening methods. Accordingly, therapeutic agents of the present disclosure include those agents identified as a MYC-driven renal cell carcinoma (RCC) therapeutic agent.

Methods of identifying a therapeutic agent that is effective against MYC-driven neoplasms will vary. Such methods may include contacting a MYC-driven neoplasm with a candidate agent; obtaining a candidate lipogenesis profile for the MYC-driven neoplasm following the contacting; identifying the candidate agent as a MYC-driven neoplasm therapeutic agent when the candidate lipogenesis profile indicates decreased lipogenesis as compared to a control lipogenesis profile. Such methods may be performed in vitro or in vivo. In some instances, such a method may employ a MYC-driven neoplasm that is a mammalian (e.g., human, mouse, rat, etc.) MYC-driven neoplasm xenograft.

Lipogenesis profiles useful in such methods of screening may be obtained by any convenient method, including e.g., those described above, such as e.g., MS, such as e.g., DESI-MSI. Lipogenesis profiles obtained from cellular samples contacted with one or more candidate agents may be compared to a control. Useful controls include e.g., control lipogenesis profiles, including e.g., lipogenesis profiles of the MYC-driven neoplasm prior to the contacting; a lipogenesis profile of noncancerous cells; a lipogenesis profile of a lipogenesis independent neoplasm; a lipogenesis profile of a non-MYC driven neoplasm; and the like. In some instances, methods of screening may include measuring an activity of one or more lipogenesis genes (e.g., ACLY, ACACA, FASN, SCD, etc.) or a protein expressed therefrom in a MYC-driven neoplasm, including where such measuring is performed following contacting with the candidate agent.

In some instances, a subject method of screening may include assessing the effectiveness of the candidate agent, including e.g., measuring one or more aspect of a neoplasm contacted with the agent. For example, in some instances, the screening method may include identifying the candidate agent as a MYC-driven neoplasm therapeutic agent when the MYC-driven neoplasm regresses following contacting with the agent. In some instances, a measured decrease in lipogenesis may be observed, including e.g., a decrease in glycerophosphoglycerols. In some instances, a measured increase in lipogenesis may be observed, including e.g., an increase in in glycerophosphoinositols. Such increases and decreases may be compared to one or more controls.

In some instances, methods of treating a subject as described herein may include utilizing one or more agents identified in a screening method, as described above. Including e.g., where the method is a method of treating a subject for a MYC-driven neoplasm, where the method includes administering to the subject an effective amount of a MYC-driven neoplasm therapeutic agent identified from a screening, including e.g., one or more of the screening methods described herein.

### Kits

Also provided are kits for use in the subject methods. The subject kits may include any combination of components (e.g., reagents, cell lines, etc.) for performing the subject methods, such as e.g., methods of treating a subject for a neoplasm and/or methods of identifying a MYC-driven neoplasm therapeutic agent. The subject kits may include a combination of agents for use in treating a subject, i.e., a “treatment kit”. The subject kits may include cell lines (e.g., cell lines for use in screening) which may include neoplastic cell lines (e.g., tumor cell lines, cancer cell lines, etc.).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit or cell line(s), in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example 1

### MYC Mediated Lipogenesis is Essential for Neoplastic Growth

### Materials and Methods

Cell culture conditions. P493-6 cells are cultured in Gibco's RPM11640 media under normal culture conditions of 20% O₂, 4% CO₂, and 37° C. P493 cells are suspension cells, and they are kept at 0.2-1.8 million cells/ml density to avoid confluency. In order to turn off Myc, 1 μg/ml of tetracycline is applied to the media for 48 hours to completely suppress Myc. Mouse-derived renal carcinoma line E28 and human RCC 786-O cell line are cultured for in vitro experiments under normal culture conditions of 20% O₂, 4% CO₂, and 37° C. 786-O and E28 cells are maintained in Dulbecco's Modified Eagle Medium (DMEM), which is supplemented with 10% fetal bovine serum (FBS), 1% L-glutamine, 1% sodium pyruvate, 1% nonessential amino acids, and Antibiotic-Antimycotic. Trypsin-EDTA is used to passage E28 and 786-O cells. All cell culture reagents are purchased from Gibco® (Thermo Fisher Scientific Inc.).

Small molecule inhibitors. The small molecule, TOFA (Sigma), diluted in dimethyl sulfoxide (DMSO) to the desired concentrations is added to cell culture medium to achieve inhibition of ACACA. TOFA is an allosteric inhibitor of ACACA, the enzyme which catalyzes the rate-limiting step of fatty acid synthesis. Various concentrations of TOFA are administered to cells in culture over time to assess dose-response suppression of proliferation. For in vivo treatment of mice, 25 mg/kg TOFA is administered daily by intraperitoneal injections (IP). The stock TOFA is diluted in DMSO to 10 mg/ml, such that the final injected volume is around 50 μl since the mice weigh about 20 g.

Cell counting. A volume of cells is removed from culture medium and mixed with an equal volume of 0.4% Trypan blue stain. Then 10 μl hemocytometer for cell counting. Viable cell counts are used as a measure of cell proliferation.

RNA extraction & cDNA synthesis. RNA extraction from 2×10⁷ cells is done using the Qiagen RNEasy Extraction kit. RNA quality and concentration are assessed by a spectrophotometer, the Thermo Scientific's Nanodrop 3300. cDNA is then synthesized from 0.4 μg of the extracted RNA using Qiagen cDNA reverse transcription kit. The cDNA is then stored at −20° C.

Primers & qRT-PCR. Primers are designed to span intron-exon junction, using PrimerQuest online software that is available from www.idt.com. Primer specificity is then tested using BLAST. Real-time PCR is performed in 96-well plates on an ABI Biosystems Thermo Cycler 7500. All primers are detected by using SYBR Green as fluorophore. Reactions are carried out in 20 μl that contained 1.5 μl cDNA, 0.5 μM forward and reverse primers and 8 μl water and 10 μl of 2× SYBR Green master mix (Applied Biosystems). Amplification cycle is as follows: 95° C. for 3 min, 35 cycles of 95° C. for 10 s, 63° C. for 30 s, 72° C. for 30 s and a final extension at 72° C. for 5 min. At the end of the amplification cycles, a dissociation curve is obtained to verify non-specific amplification. The cycler software yields threshold cycle (Ct) number for each gene; Ct is the number of cycles required to reach the threshold fluorescence. The Ct values are exported into Excel for statistical analysis.

Nuclear Run-On Assay. Cells are harvested and washed with cold Phosphate Buffer Saline (PBS). 10 ml of cold Cell Lysis Buffer is added to resuspend each nuclei pellet with 100 μl of Nuclei Resuspension Buffer to isolate the nuclei. Then Nuclear Run-On buffer is added for 30 min, then incubated at 30° C. and reset temperature at 37° C. 200 U of DNase I (10 U/μl, Roche Applied Sciences) is added to each reaction and incubated for 20 min at 37° C. 400 U of Proteinase K (20 mg/ml, Ambion, premixed with 10% sodium dodecyl sulfate at 3:1) is added and then incubated for 15 min at 37° C. Then extraction of total RNA is done using the Qiagen RNA extraction kit. Dynabeads and immobilized biotin-labeled nascent RNA are washed to prepare for first-strand and second-strand cDNA synthesis. This is then followed by cDNA purification, cRNA synthesis, cRNA purification, and array hybridization.

Western blot. Proteins from cell lysate are obtained by lysing the cells in M-PER (BioRad), detergent-containing lysis buffer. Quantitation is done using the BCA-kit (BioRad). 50 μg of protein is then loaded into precast gels (BioRad) and run under 150 mV for 1 hour. After completion, dry transfer is done using the iBlot (Invitrogen) machine for 7 min. Membrane is then blocked using Cassein Blocker solution (Thermo Scientific) for one hour. This is followed by overnight incubation of membranes in primary antibodies in 4° C. Washes are done using the SnapID vacuum system (Millipore) 3× between primary and secondary antibodies. ECL reagent (GE Healthcare) is then used to obtain and develop the films.

Chromatin Immunoprecipitation (ChIP). Chromatin immunoprecipitation (ChIP) is performed on P493 cells as using the Imprint ChIP kit from Sigma-Aldrich (Sigma CHP1) following the conditions described in our previous paper. The P493 cells are treated with formaldehyde (1% final concentration) for 10 min. Chromatin from 10 million cells is then incubated in 1 μg antibody. The complexes are pulled down with StaphA cells and washed. This is then followed by the reversed cross-linking. Antibodies used for ChIP are anti-MYC (Santa Cruz cat #sc-764, Epitomics rabbit monoclonal cat #1472-1), IgG control (normal rabbit IgG, Santa Cruz cat #sc-2027).

De Novo lipogenesis assay by NMR. 100 μl of cells is utilized for lipid analysis. Pellet is resuspended in 18 mL of fresh medium that contains 10% dialyzed Fetal Bovine Serum, 2.0 mM [1-13C] glucose, 8.0 mM unenriched glucose, 3.0 mM [U-13C] glutamine and no unenriched glutamine. Resuspended cells are then incubated at 37° C. for 14 hours, harvested, and quickly frozen in liquid nitrogen for about 10 seconds and stored at −80° C. 2 mL of cold (4° C.) methanol is added to the cell pellet in a 50 mL conical centrifuge tube, followed by 1 mL of chloroform to the methanol. The mixture is sonicated with 1 mL of high purity water in the mixture. The sample is sonicated again, then centrifuged to separate the aqueous and chloroform phases. The chloroform phase is removed and the methanol-water phase is transferred back to the original 50 mL conical centrifuge tube and with the addition of 1 mL of chloroform. This sonication and separation are repeated two more times. Finally, it is added to 600 mg of CDCl₃ and 13 mg (1 drop) of CH₂Cl₂. The chloroform-lipid solution is transferred to an NMR tube to equilibrate to room temperature for at least one hour.

Glucose & Oleate incorporation assay by Mass Spectrometry. Media are aspirated, cells are rinsed twice with 2 mL of Phosphate Buffered Saline in room temperature, 1 mL of 50:50 MeOH/H₂O solution is added with 0.1 M HCl at −20° C. Then the resulting liquid and cell debris are scraped into a microfuge tube. 0.3 M KOH is then added, and the mix is incubated at 80° C. for 1 h to saponify fatty acids, acidified with 0.1 mL of formic acid, extracted twice with 1 mL of hexane, N₂ dried. Finally, it is reconstituted into 1:1:0.3 MeOH:chloroform:H2O (1 mL of solvent per 2 μL of packed volume for cells, and 2 mL of solvent total for the medium samples) for liquid chromatography-mass spectrometry (LC-MS) analysis. Separation is done by reversed-phase ion-pairing chromatography on a C8 column coupled to negative-ion mode, full-scan LC-MS at 100,000 resolving power (stand-alone Orbitrap (Exactive); Thermo Fischer Scientific).

Desorption Electrospray Ionization Mass Spectrometry Imaging (DESI-MSI). DESI-MSI is used for generating two-dimensional chemical maps of various tissues sections in order to assess the lipid profile of an organ. Tissue sections of 16 μm thick are imaged by this method using a lab-built DESI-MSI source coupled to an LTQ-Orbitrap XL mass spectrometer (Thermo Fisher Scientific, MA), and the mass spectra are acquired in negative ion mode using the Orbitrap as the mass analyzer at 60,000 resolving power. We use tissue nondestructive solvent dimethylformamide: acetonitrile to desorb and ionize phospholipids. This allows a subsequent histological evaluation of the same specimen to ensure a correct interpretation of lipid signal collected from various pathological regions. At least three tissue samples for each disease model and state are imaged. All tissues are histopathologically evaluated after imaging using standard hematoxylin and eosin (H&E) staining, to ensure a correct interpretation of lipid signals.

The software !mgGenerator (freeware, version 1.3) is used for converting raw mass spectra files into 2D images. Spatially accurate ion images are assembled using BioMap software. Tandem MS analyses are performed using both the Orbitrap and the linear ion trap for mass analysis to confirm lipid identity. The LipidMaps database is employed to assist in lipid identification.

### Results

We present a comprehensive overview of how the MYC oncogene controls lipogenesis to promote tumorigenesis. We find that MYC globally regulates the expression of key genes and their resulting proteins and metabolites in lipogenesis for human and murine cell lines as well as for transgenic mouse models. By gene expression analysis, nuclear run-on, ¹³C-isotope labeling, and chromatin immuno-precipitation, MYC in conjunction with a Sterol Regulatory Element-Binding Protein 1 (SREBP1) was found to induce the nonlinear expression of fatty acid biosynthesis pathway genes including Acetyl-CoA Carboxylase A (ACACA), Fatty Acid Synthase (FASN), and Stearoyl-CoA Desaturase (SCD) and to drive fatty acid chain elongation from glucose and glutamine. Desorption electrospray ionization mass spectrometric imaging (DESI-MSI) from tissue samples of several conditional transgenic mouse models identified in vivo metabolic changes in simple fatty acids and in complex phospholipids induced by MYC. Thus, MYC not only increased oleate abundance in multiple tumors, but also notably elevated the abundances of phosphatidylglycerols with elongation of fatty acid side chains and concomitant suppression of phosphatidylinositol abundances in a transgenic renal cell carcinoma model. Long, polyunsaturated fatty acids were also notably higher in abundance with prolonged MYC activation. Strong correlation between these in situ changes and elevation of gene expressions of cytidinediphosphate diacylglycerol pathway genes as well as fatty acid elongase genes were found. By administering cerulenin and 5-tetradecyl-oxy-2-furoic acid (TOFA), which are known to inhibit fatty acid metabolism, we report that MYC-induced tumorigenesis was blocked, metabolic changes were suppressed, and tumors regressed in xenograft and primary mouse models. These results expose the vulnerability of MYC-induced cancers to inhibition of fatty acid synthesis, which might serve as a therapeutic approach.

In normal metabolism the key regulators of fatty acid synthesis are well understood. In cancer, regulation of lipogenesis is more complicated. We considered that MYC may regulate a more complex lipogenesis which would be essential to cancer cell division and growth, such as glycerophospholipid biosynthesis. Glycerophospholipids (GP) are long amphiphilic molecules comprised of two fatty acid chains and a phosphate group ester-linked to a glycerol moiety, whereas another substituent is attached to the phosphate group. The chemical nature of the phosphate group substituent divides GPs into several molecular classes, whereas various combinations of fatty acid chain length, and saturation give rise to many different molecules within each class. GPs play an essential role in biomass growth because they supply building blocks and provide suitable physical properties for cell and organelle membranes, act as cell signaling mediators, and are implicated in energy metabolism.

We examined these pathways both in human and mouse tumor-derived cell lines and in in vivo transgenic mouse models using genome-wide expression profiling, nuclear run-on profiling, ChIP analysis, and DESI-MSI. DESI-MSI enabled us to monitor both simple and complex lipids in situ without compromising metabolic homeostasis of the examined organ. In this sense this technology is superior to many other metabolomic platforms. DESI-MSI provides a detailed map of molecular distribution for each detected metabolite in ambient conditions and without the use of matrix substrates, labeling, or molecule pre-identification. DESI-MSI also has a superior capability to detect fatty acids and complex glycerophospholipids.

MYC Regulates Expression of Lipogenesis. MYC has been suggested to regulate specific genes in lipogenesis. We performed genome-wide expression profiling, nuclear run-on profiling, and ChIP analysis in the human P493-6 B-cell line that are engineered to conditionally overexpress human MYC gene regulated by the Tet System. MYC induction increased mRNA expression of fatty acid synthesis genes including: ACLY, ACACA, FASN, and SCD (FIG. 1a) in four of our MYC-induced cell systems while also showing that the catabolic fatty acid oxidation genes are suppressed by MYC (FIG. 1b). MYC bound to the promoters by ChIP of fatty acid synthesis genes (FIG S1a, for raw data: S1b) and mevalonate & cholesterol pathway genes (FIG. S1c) including: HMGCR and DHCR7, (FIG. S1d), as measured by qPCR). Generally, MYC induces expression of lipogenesis genes in both tumor-derived cell lines from transgenic mouse models of MYC-induced T-cell lymphoma (T-ALL), lung cell carcinoma, renal cell carcinoma (RCC), hepatocellular carcinoma (HCC) as well as in in vivo transgenic tumors from both HCC and RCC, including the genes: Acly, Acaca Fasn, Scd (FIG. 1c), Hmgcr, and Dhcr7 (FIG. S1d). MYC binds to the promoters of many of the fatty acid synthesis genes (FIG. 2a), as measured in BCL by ChIP (FIG. 2b) and validated by Nuclear Run-On (FIG. 2c) and qPCR (FIG. S1e). MYC binding is not only on fatty acid synthesis genes, but also on glycolysis and glutaminolysis genes (FIG. 2d). Therefore, MYC binds to the promoters and induces the expression of many lipogenesis genes.

MYC requires SREBP1 to induce Expression of Fatty Acid Synthesis. SREBP1 regulates the fatty acid synthesis pathway, that in turn is regulated by SCAP and INSIG. MYC induced both SREBP1 and SCAP in murine HCC lines EC4 and HCC3-4 by qPCR (FIG. 3a). The siRNA knockdown of Srebf1 (FIG. 3b) in EC4 and HCC3-4 reduced MYC induced expression of ACLY, ACACA, FASN, SCD (FIG. 3b). Similarly inactivation of MYC leads to reduced Srebf1-induced expression of the aforementioned fatty acid synthesis genes (FIG. 3b). Therefore, MYC regulates the expression of SREBP1 and they collaborate to regulate fatty acid synthesis genes.

MYC induction of Glucose and Glutamine Metabolism Contributes to Lipogenesis. MYC is known to regulate glucose and glutamine metabolism. Upon induction, MYC is shown to bind to and transcribe glycolytic (FIG. 2d), glutaminolytic (FIG. 2d), fatty acid and cholesterol biosynthetic genes (FIG. S2a) which are confirmed by qPCR (glycolysis genes: FIG. S2b, glutaminolysis genes: FIG. S2c, and fatty acid synthesis genes FIG. S2d) temporally in sequential manner, in P493-6 by ChIP (FIG. 1b). Correspondingly, after 24 hours of MYC induction, 80% of lipid carbons were derived from glucose and about 20% were from glutamine in P493-6 by metabolic tracing (FIG. 4a) with DESI-MSI in multiple models (FIG. 4b) that is confirmed by tracing [1-13C]-glucose by mass spectrometry (FIG. 4c), and confirmed by NMR (FIG. 4d, FIG. S3a for high resolution). Furthermore, we notice there is a relative increase of unsaturated fatty acids compared to saturated fatty acids (for short fatty acids: FIG. S3b, and long fatty acids: FIG. S3c) which is consistent with MYC's induction of the desaturase, SCD (FIG. 1c). Thus, MYC coordinates glycolysis glutaminolysis, and fatty acid synthesis.

MYC Regulates Fatty Acid Synthesis and Production of Phospholipids in vivo. To examine if MYC induces lipogenesis in vivo, we used DESI-MSI that provides a histological level portrait of organ metabolism, and our conditional transgenic models in which we could induce MYC-driven tumors and reverse them by MYC inactivation. We found that MYC increased relative abundances of unsaturated fatty acids, and specifically oleic acid FA(18:1) m/z 281.248, the end product of the fatty acid synthesis pathway described above (FIG. 1a). Increase in relative abundance of oleate was detected across various MYC-tumor bearing tissues (FIG. 4b, for fold change: FIG. S4a). Second, MYC induction significantly modified relative abundances of complex glycerophospholipids in RCC, LCC, HCC, and T-LL (FIG. 5a). The vast majority of these changes were reversible upon MYC inactivation (FIG. 5b). Hence, MYC regulates lipogenesis products in vivo.

MYC Promotes in Vivo Fatty Acid Elongation and Desaturation and Differentially Regulates GPs with Time. To take a closer look at changes in lipids occurring with time during MYC activation and subsequent inactivation, we chose to perform DESI-MSI analysis in MYC-induced RCC transgenic model, which allows both uniform induction of MYC expression and preservation of histologic architecture. We performed our analysis under conditions in which a wide variety of complex glycerophospholipids of different classes could be detected. We found that the most significant MYC-induced dynamic changes occurred in relative and total abundances of glycerophosphoglycerols (PGs), GPs in which the phosphate group substituent is glycerol (FIG. 6a). PGs are products of complex lipogenesis: they are formed from phosphatidic acid (PA), which is synthesized by the addition of two fatty acids to glycerol 3-phosphate that in turn is formed primarily from glycolysis (FIG. 6b).

Overall, MYC markedly increased the abundances of various PGs in tissue with time of activation as detected by DESI-MSI (FIG. 7a, for other PGs: FIG. S4b and FIG. S4c, for fold change: FIG. S4d and FIG. S4e). By qPCR we identified six genes involved in PG synthesis (FIG. 6b) that were progressively overexpressed with MYC activation and then suppressed with MYC inactivation (FIG. 7b). In particular, a very prominent induction was observed for cytidinediphosphate diacylglycerol (CDP-DAG) pathway genes: Pgs1 (phosphatidylglycerophosphate (PGP) synthase, converts CDP-DAG to PGP), and Ptpmt1 (PGP phosphatase, dephosphorylates PGP to generate PGs) (FIG. 7b). MYC induction also upregulated Agpat1 and Cds1, that catalyze prerequisite steps to CDP-DAG conversion to PGs: the transformation of lysophosphatidic acid (LPA) to PA and subsequently to CDP-DAG (FIG. 6b).

Intriguingly, not all subtypes of PGs identified by DESI-MSI increased in a similar manner with MYC activation. PGs with shorter side fatty acid chains, which comprised 18 carbons or less as identified by tandem MS, increased initially in their relative abundances, and then decreased following MYC induction (FIG. 7a, for other lipids: FIG. S4b, for fold change: FIG. S4d). In particular, m/z 745.501, identified as PG (18:2/16:0), showed highest increase in relative intensities at 10 days of MYC activation, which decreased at 15 days of MYC activation (FIG. 7a). Similar trend was observed for lipid species at m/z 747.516, m/z 773.532, m/z 775.547, and m/z 771.517 (FIG. S4b), identified as PG(18:1/16:0), PG(18:1/18:1), PG(18:0/18:1), PG(18:2/18:1) respectively by tandem MS (Table 1). Note that the FA chains compositions are assigned to these molecules based on the most prevalent tandem MS fragmentation pattern, and that all assignments have an absolute mass error of less than 4 ppm.

Concomitantly with the decrease in these species' abundances, we observed an increase in relative intensities of long PGs with at least one side chain longer than 20 carbons (FIG. 7a, for other lipids: FIG. S4c, for fold change: FIG. S4e). Specific molecular ions that followed this pattern were at m/z 865.500, m/z 819.517, m/z 867.519, m/z 821.534, m/z 841.500, and m/z 817.500 (FIG. S5b), which were identified as PG(22:6/22:6), PG(18:1/22:6), PG(22:6/22:5), PG(18:1/22:5), PG(20:4/22:6), PG(18:2/22:6) respectively (Table 1). In particular, m/z 865.500 and m/z 867.519, identified as PG(22:6/22:6) and PG(22:6/22:5) showed a marked temporal increase in the total abundances with the highest increase in average relative intensity at 15 days of MYC activation (FIG. S4c, for fold change: FIG. S4e, for mass spectra: FIG. 6a). Those changes were reversible with MYC inactivation (FIG. S4c, for fold change: FIG. S4e, for mass spectra: FIG. 6a).

A plausible explanation for this differential PG signature is that with time MYC induction drives toward a formation of long, polyunsaturated fatty acids, which are being preferentially incorporated into de novo synthesized PGs through acyl chain remodeling. Consistent with this, MYC induced mRNA expression of the fatty acid elongases Elovl2 and Elovl6, that add 2 carbons to long fatty acid chains, as measured by qPCR (FIG. 7c).

In contrast, MYC suppressed another GP species, glycerophosphatidylinositols (PIs) as measured by DESI-MSI (FIG. S5a; for fold change: S5b). These changes were again reversible with MYC inactivation. PIs have inositol as phosphate group substituent and they are competitively synthesized with PGs through a common pool of CDP-DAG (FIG. 6b). Significant induction in PGS1 and PTPMT1 promote preferential CDP-DAG conversion to PGs which shunts metabolites away from PI synthesis.

Hence, MYC markedly upregulates PG synthesis and increases the production and incorporation of long unsaturated FAs as shown on in situ lipid level and on mRNA level. The induction of CDP-DAG synthesis and elongases is consistent with the binding data in P493 (FIG. S6a, S6b).

Induction of PG synthesis with MYC upregulation is consistently observed across various tissues and can be considered a hallmark signature of MYC-aberrated metabolism (FIG. 5a). These changes are reversible with MYC inactivation (FIG. 5b). The changes in PIs are less consistent. Thus, PIs are not suppressed, but rather elevated in LCC, possibly due to the presence of lung surfactant rich in PGs and other GPs.

MYC Regulation of Lipogenesis is Essential for Cancer Initiation and Progression. We examined how the inhibition of lipogenesis influenced MYC-induced tumor cell growth in vitro and in vivo (FIG. S7a). First, transgenic tumor-derived cell lines from RCC (E28), HCC (EC4) and T-ALL (4188) and human lymphoma B-cell line (P493-6) had their growth inhibited in vitro in a dose-dependent manner upon the inhibition of fatty acid synthesis by cerulenin, a potent irreversible inhibitor of FASN (FIG. S7b), or by 5-tetradecyl-oxy-2-furoic acid (TOFA), ACACA inhibitor in lymphoma B-cell line (FIG. S7c) and in kidney cell line (FIG. S7d), as measured by total live cell counts, which are partially rescuable by the addition of oleate (FIG. S7e).

Second, TOFA treatment before tumor onset in transgenic MYC-induced RCC prevented tumor growth (FIG. 8a), and TOFA treatment after tumor onset induced tumor regression as measured by kidney weight and size (FIG. 8b) and by histological examination (FIG. 8c). This regression can be tracked by DESI-MSI (FIG. 9a). Because Cerulein inhibits FASN, there can be accumulation of Malonyl-CoA which in turn inhibits also fatty acid oxidation. On the other hand, inhibition of ACACA would not cause accumulation of malonyl CoA and inhibition of fatty acid oxidation. To avoid inhibition of fatty acid oxidation and to specifically inhibit fatty acid synthesis, only TOFA was used in our in vivo models. Third, the human RCC tumor derived cell line, 786-0, when treated in vitro or in vivo when grown as a xenograft in NSG mice exhibited tumor regression upon treatment with TOFA (FIG. 9b). Fourth, by DESI-MSI, TOFA was found to block PGs and PIs during tumor initiation and regression in transgenic MYC induced RCC (FIG. 9a, for other lipids: FIG. S8a, S8b) and human RCC xenograft (FIG. 9b, for other lipids: FIG. S8c, S8d). Thus, TOFA inhibition of fatty acid synthesis blocks MYC induced tumor growth in vitro and in vivo in mouse and human tumor-derived cell lines and in an autochthonous transgenic mouse or human xenograft.

### Discussion

We report that MYC regulates lipogenesis including: fatty acid synthesis, cholesterol biosynthesis and glycerophospholipid metabolism (FIG. 10), via collaboration with SREBP1. Importantly, through DESI-MSI we performed analysis of lipogenesis products to in situ identify MYC-induced lipid aberrations. MYC requires lipogensis and specifically fatty acid synthesis to maintain tumorigenic growth in vitro and in vivo. Our results have implications for how MYC coordinates metabolism and growth control, suggest a novel therapeutic vulnerability for MYC driven tumors and provide evidence that DESI-MSI can be employed as a tool to estimate lipid metabolism in tumorigenesis and assess drugs that target and prevent cancers.

Our results suggest that MYC globally regulates lipogenesis following induction of glycolysis and glutaminolysis. Through RNAseq, ChlPseq, and Nuclear Run-ON assays, we found that MYC amplifies gene expression non-linearly and associates with groups of genes seemingly in an orderly fashion to stimulate metabolism and biomass accumulation. Upon induction of MYC in a human lymphoma model P493-6, transgenic murine liver, kidney, and lung cancer models, we found that MYC induces fatty acid synthesis genes. The induction of fatty acid synthesis genes kinetically followed the induction of glycolytic and glutaminolytic genes, suggesting a possible temporal regulation of metabolic genes in sequence. These observations reveal the complexity of MYC-dependent gene expression amplification, which does not result simply from amplifying all genes that are expressed at basal. Rather, our time-series studies indicate that MYC binds metabolic genes in a temporally sequential manner. As we discuss below, this is likely determined by other transcription factors, including SREBPs that we demonstrate cooperate with MYC to regulate lipogenic genes.

We found MYC collaborates with SREBP1 to synergistically activate the fatty acid synthesis genes. MYC with Srebp2 cooperates to induce the expression of mevalonate and cholesterol biosynthetic genes. Further, MYC activates SREBP1 by directly inducing its transcription as well as by inducing the positive regulators, SCAP and repressing the negative regulator, INSIG. Our analysis of ChIP data suggest that Mxd1 and Mxd4, which are known to bind Mlx, competitively displace Mlxip from Mix and inhibit the activity of Mlxip. Accordingly, the results suggest that MYC globally regulates lipogenesis but with different partners.

By DESI-MSI, we were able to uniquely visualize the spatio-temporal lipid modifications to identify neoplastic disease, follow disease progression and predict and monitor tumor regression in vivo. We showed that MYC's regulation of lipogenesis results in a distinct phospholipid signature in human and mouse tumors, and across tumor types including: T-ALL, B-cell lymphoma, LCC, HCC and RCC, that distinguishes normal from malignant tissue.

First, the lipid signature included broad increase in PGs but some different changes in PIs. PG is a minor phospholipid component of many intracellular membranes, accounting for less than 1% of total phospholipids in most nonneoplastic mammalian tissues. PGs are precursors of cardiolipins, polyglycerolphospholipids primarily localized at inner membranes of mitochondria and required for the activity of many mitochondrial enzymes and mitochondrial membrane integrity. PGs are also potential activators of protein kinase C family, and in particular nuclear protein kinase C-β_{II}. The increase in PGs is consistent with induction of mitochondrial biogenesis orchestrated by MYC. The decrease in PIs which share the common synthetic pathway with PGs through CDP-DAG (FIG. 6b) is seen in most organs, but not in lung. In lung PG pool is much more abundant compared to other tissues because PGs are the major component of pulmonary surfactant. Pulmonary surfactant also comprises PIs and other phospholipids.

Second, we found there was a distinct temporal sequence to these lipid changes, that we could identify in our transgenic model of MYC-induced RCC, that were associated with each stage of tumorigenesis. Specifically, with time MYC globally elevated PGs with longer, polyunsaturated acyl tails, possible through acyl chain remodeling. We show that fatty acid elongases are induced by MYC.

Third, upon MYC inactivation in a tumor, the inhibition of lipogenesis was associated with specific changes in the lipid signature. Correspondingly, upon therapeutic inactivation of fatty acid synthesis by TOFA, we identified a lipid signature associated with therapeutic response. Thus, DESI-MSI and measurement of lipid signature can be used to diagnose cancer and identify novel therapeutics.

Finally, our results suggest that MYC induced tumors are dependent on lipogenesis and specifically fatty acid synthesis. We show that inhibition of ACACA alone induced proliferative arrest and apoptosis of many different MYC associated tumors. Further, in vivo inhibition of ACACA, prevented MYC from inducing tumorigenesis in vivo and could induce tumor regression in established tumors using an autochthonous transgenic mouse model of MYC-induced RCC. Our results suggest that MYC induced human tumors may be highly sensitive to inhibition of fatty acid synthesis. There is an emerging general model, that MYC orchestrates the orderly activation of glycolysis, glutaminolysis and fatty acid synthesis, providing a means for the balanced acquisition of nutrients and stoichiometric production of cellular biomass. For a normal cell, this is essential to enable the coordination between coordinating the need for energy metabolism and generating building blocks for biomass generation. MYC's regulation of lipid metabolism similarly is required to coordinate the respective requirements for energy, signaling molecules and membrane production. For a cancer cell, MYC overexpression provides the ability to maximize unrestrained growth but at the expense of a remarkable vulnerability to the inhibition of key regulators of this pathway, such as ACACA. The development of DESI-MSI enabled us to globally map the MYC specific in lipid metabolism and now will enable us to identify new drugs that target lipogenesis as a treatment for this Achilles heel of cancer.

Through examination of human tumor derived cell lines and in vivo transgenic mouse models we have shown that MYC directly regulates FA synthesis and complex GP formation at multiple steps. In turn, inhibition of lipid metabolism blocks and reverses MYC-induced tumorigenesis.

### REFERENCES

1. Wang, Z. R., Liu, W., Smith, S. T., Parrish, R. S., and Young, S. R. 1999. c-myc and chromosome 8 centromere studies of ovarian cancer by interphase FISH. Exp Mol Pathol 66:140-148.
2. Cawley, S., Bekiranov, S., Ng, H. H., Kapranov, P., Sekinger, E. A., Kampa, D., Piccolboni, A., Sementchenko, V., Cheng, J., Williams, A. J., et al. 2004. Unbiased mapping of transcription factor binding sites along human chromosomes 21 and 22 points to widespread regulation of noncoding RNAs. Cell 116:499-509.
3. Fernandez, P. C., Frank, S. R., Wang, L., Schroeder, M., Liu, S., Greene, J., Cocito, A., and Amati, B. 2003. Genomic targets of the human c-Myc protein. Genes Dev 17:1115-1129.
4. Ayer, D. E., Kretzner, L., and Eisenman, R. N. 1993. Mad: a heterodimeric partner for Max that antagonizes Myc transcriptional activity. Cell 72:211-222.
5. Baudino, T. A., and Cleveland, J. L. 2001. The Max network gone mad. Mol Cell Biol 21:691-702.
6. Brodeur, G. M., Seeger, R. C., Schwab, M., Varmus, H. E., and Bishop, J. M. 1984. Amplification of N-myc in untreated human neuroblastomas correlates with advanced disease stage. Science 224:1121-1124.
7. Cole, M. D., and McMahon, S. B. 1999. The Myc oncoprotein: a critical evaluation of transactivation and target gene regulation. Oncogene 18:2916-2924.
8. Dang, C. V. 2012. MYC on the path to cancer. Cell 149:22-35.
9. Drabkin, H. A., Bradley, C., Hart, I., Bleskan, J., Li, F. P., and Patterson, D. 1985. Translocation of c-myc in the hereditary renal cell carcinoma associated with a t(3;8)(p14.2;q24.13) chromosomal translocation. Proc Natl Acad Sci USA 82:6980-6984.
10. Shroff, E. H., Eberlin, L. S., Dang, V. M., Gouw, A. M., Gabay, M., Adam, S. J., Bellovin, D. I., Tran, P. T., Philbrick, W. M., Garcia-Ocana, A., et al. 2015. MYC oncogene overexpression drives renal cell carcinoma in a mouse model through glutamine metabolism. Proc Natl Acad Sci USA 112:6539-6544.
11. Tang, S. W., Chang, W. H., Su, Y. C., Chen, Y. C., Lai, Y. H., Wu, P. T., Hsu, C. I., Lin, W. C., Lai, M. K., and Lin, J. Y. 2009. MYC pathway is activated in clear cell renal cell carcinoma and essential for proliferation of clear cell renal cell carcinoma cells. Cancer Lett 273:35-43.
12. Taub, R., Kirsch, I., Morton, C., Lenoir, G., Swan, D., Tronick, S., Aaronson, S., and Leder, P. 1982. Translocation of the c-myc gene into the immunoglobulin heavy chain locus in human Burkitt lymphoma and murine plasmacytoma cells. Proc Natl Acad Sci USA 79:7837-7841.
13. Dalla-Favera, R., Bregni, M., Erikson, J., Patterson, D., Gallo, R. C., and Croce, C. M. 1982. Human c-myc one gene is located on the region of chromosome 8 that is translocated in Burkitt lymphoma cells. Proc Natl Acad Sci USA 79:7824-7827.
14. Blackwood, E. M., Kretzner, L., and Eisenman, R. N. 1992. Myc and Max function as a nucleoprotein complex. Curr Opin Genet Dev 2:227-235.
15. Dang, C. V., Resar, L. M., Emison, E., Kim, S., Li, Q., Prescott, J. E., Wonsey, D., and Zeller, K. 1999. Function of the c-Myc oncogenic transcription factor. Exp Cell Res 253:63-77.
16. Dang, C. V., O'Donnell, K. A., Zeller, K. I., Nguyen, T., Osthus, R. C., and Li, F. 2006. The c-Myc target gene network. Semin Cancer Biol 16:253-264.
17. Felsher, D. W., and Bishop, J. M. 1999. Reversible tumorigenesis by MYC in hematopoietic lineages. Mol Cell 4:199-207.
18. Felsher, D. W. 2004. Reversibility of oncogene-induced cancer. Curr Opin Genet Dev 14:37-42.
19. Gao, P., Tchernyshyov, I., Chang, T. C., Lee, Y. S., Kita, K., Ochi, T., Zeller, K. I., De Marzo, A. M., Van Eyk, J. E., Mendell, J. T., et al. 2009. c-Myc suppression of miR-23a/b enhances mitochondrial glutaminase expression and glutamine metabolism. Nature 458:762-765.
20. Wise, D. R., DeBerardinis, R. J., Mancuso, A., Sayed, N., Zhang, X. Y., Pfeiffer, H. K., Nissim, I., Daikhin, E., Yudkoff, M., McMahon, S. B., et al. 2008. Myc regulates a transcriptional program that stimulates mitochondrial glutaminolysis and leads to glutamine addiction. Proc Natl Acad Sci USA 105:18782-18787.
21. Laplante, M., and Sabatini, D. M. 2012. mTOR signaling in growth control and disease. Cell 149:274-293.
22. Lewis, C. A., Griffiths, B., Santos, C. R., Pende, M., and Schulze, A. 2011. Regulation of the SREBP transcription factors by mTORC1. Biochem Soc Trans 39:495-499.
23. Le, A., Lane, A. N., Hamaker, M., Bose, S., Gouw, A., Barbi, J., Tsukamoto, T., Rojas, C. J., Slusher, B. S., Zhang, H., et al. 2012. Glucose-independent glutamine metabolism via TCA cycling for proliferation and survival in B cells. Cell Metab 15:110-121.
24. Metallo, C. M., Gameiro, P. A., Bell, E. L., Mattaini, K. R., Yang, J., Hiller, K., Jewell, C. M., Johnson, Z. R., Irvine, D. J., Guarente, L., et al. 2012. Reductive glutamine metabolism by IDH1 mediates lipogenesis under hypoxia. Nature 481:380-384.
25. Wise, D. R., Ward, P. S., Shay, J. E., Cross, J. R., Gruber, J. J., Sachdeva, U. M., Platt, J. M., DeMatteo, R. G., Simon, M. C., and Thompson, C. B. 2011. Hypoxia promotes isocitrate dehydrogenase-dependent carboxylation of alpha-ketoglutarate to citrate to support cell growth and viability. Proc Natl Acad Sci USA 108:19611-19616.
26. Griffiths, B., Lewis, C. A., Bensaad, K., Ros, S., Zhang, Q., Ferber, E. C., Konisti, S., Peck, B., Miess, H., East, P., et al. 2013. Sterol regulatory element binding protein-dependent regulation of lipid synthesis supports cell survival and tumor growth. Cancer Metab 1:3.
27. Swinnen, J. V., Brusselmans, K., and Verhoeven, G. 2006. Increased lipogenesis in cancer cells: new players, novel targets. Curr Opin Clin Nutr Metab Care 9:358-365.
28. Edmunds, L. R., Sharma, L., Kang, A., Lu, J., Vockley, J., Basu, S., Uppala, R., Goetzman, E. S., Beck, M. E., Scott, D., et al. 2015. c-Myc programs fatty acid metabolism and dictates acetyl-CoA abundance and fate. J Biol Chem 290:20100.
29. Morrish, F., Noonan, J., Perez-Olsen, C., Gafken, P. R., Fitzgibbon, M., Kelleher, J., VanGilst, M., and Hockenbery, D. 2010. Myc-dependent mitochondrial generation of acetyl-CoA contributes to fatty acid biosynthesis and histone acetylation during cell cycle entry. J Biol Chem 285:36267-36274.
30. Schug, Z. T., Peck, B., Jones, D. T., Zhang, Q., Grosskurth, S., Alam, I. S., Goodwin, L. M., Smethurst, E., Mason, S., Blyth, K., et al. 2015. Acetyl-CoA synthetase 2 promotes acetate utilization and maintains cancer cell growth under metabolic stress. Cancer Cell 27:57-71.
31. Wang, C., Xu, C., Sun, M., Luo, D., Liao, D. F., and Cao, D. 2009. Acetyl-CoA carboxylase-alpha inhibitor TOFA induces human cancer cell apoptosis. Biochem Biophys Res Commun 385:302-306.
32. Shao, W., and Espenshade, P. J. 2014. Sterol regulatory element-binding protein (SREBP) cleavage regulates Golgi-to-endoplasmic reticulum recycling of SREBP cleavage-activating protein (SCAP). J Biol Chem 289:7547-7557.
33. Sun, Y., He, W., Luo, M., Zhou, Y., Chang, G., Ren, W., Wu, K., Li, X., Shen, J., Zhao, X., et al. 2015. SREBP1 regulates tumorigenesis and prognosis of pancreatic cancer through targeting lipid metabolism. Tumour Biol.
34. Carroll, P. A., Diolaiti, D., McFerrin, L., Gu, H., Djukovic, D., Du, J., Cheng, P. F., Anderson, S., Ulrich, M., Hurley, J. B., et al. 2015. Deregulated Myc Requires MondoA/MIx for Metabolic Reprogramming and Tumorigenesis. Cancer Cell 27:271-285.
35. Santinon, G., Pocaterra, A., and Dupont, S. 2016. Control of YAP/TAZ Activity by Metabolic and Nutrient-Sensing Pathways. Trends Cell Biol 26:289-299.
36. Sorrentino, G., Ruggeri, N., Specchia, V., Cordenonsi, M., Mano, M., Dupont, S., Manfrin, A., Ingallina, E., Sommaggio, R., Piazza, S., et al. 2014. Metabolic control of YAP and TAZ by the mevalonate pathway. Nat Cell Biol 16:357-366.
37. Fresno Vara, J. A., Casado, E., de Castro, J., Cejas, P., Belda-Iniesta, C., and Gonzalez-Baron, M. 2004. PI3K/Akt signalling pathway and cancer. Cancer Treat Rev 30:193-204.
38. Huang, X. F., and Chen, J. Z. 2009. Obesity, the PI3K/Akt signal pathway and colon cancer. Obes Rev 10:610-616.
39. Willems, L., Tamburini, J., Chapuis, N., Lacombe, C., Mayeux, P., and Bouscary, D. 2012. PI3K and mTOR signaling pathways in cancer: new data on targeted therapies. Curr Oncol Rep 14:129-138.
40. Liu, Y., Wang, W., Shui, G., and Huang, X. 2014. CDP-diacylglycerol synthetase coordinates cell growth and fat storage through phosphatidylinositol metabolism and the insulin pathway. PLoS Genet 10:e1004172.
41. Eberlin, L. S., Gabay, M., Fan, A. C., Gouw, A. M., Tibshirani, R. J., Felsher, D. W., and Zare, R. N. 2014. Alteration of the lipid profile in lymphomas induced by MYC overexpression. Proc Natl Acad Sci USA 111:10450-10455.
42. Perry, R. H., Bellovin, D. I., Shroff, E. H., Ismail, A. I., Zabuawala, T., Felsher, D. W., and Zare, R. N. 2013. Characterization of MYC-induced tumorigenesis by in situ lipid profiling. Anal Chem 85:4259-4262.
43. Fan, J., Chen, Y. C., Watkins, T., Dang, C. V., Gorospe, M., and Cheadle, C. 2012. Array-based nuclear run-on analysis. Methods Mol Biol 809:505-517.
44. Ji, H., Wu, G., Zhan, X., Nolan, A., Koh, C., De Marzo, A., Doan, H. M., Fan, J., Cheadle, C., Fallahi, M., et al. 2011. Cell-type independent MYC target genes reveal a primordial signature involved in biomass accumulation. PLoS One 6:e26057.
45. Zeller, K. I., Zhao, X., Lee, C. W., Chiu, K. P., Yao, F., Yustein, J. T., Ooi, H. S., Orlov, Y. L., Shahab, A., Yong, H. C., et al. 2006. Global mapping of c-Myc binding sites and target gene networks in human B cells. Proc Natl Acad Sci USA 103:17834-17839.
46. Eberlin, L. S., Ferreira, C. R., Dill, A. L., Ifa, D. R., Cheng, L., and Cooks, R. G. 2011. Nondestructive, histologically compatible tissue imaging by desorption electrospray ionization mass spectrometry. Chembiochem 12:2129-2132.
47. Schuhmacher, M., Staege, M. S., Pajic, A., Polack, A., Weidle, U. H., Bornkamm, G. W., Eick, D., and Kohlhuber, F. 1999. Control of cell growth by c-Myc in the absence of cell division. Curr Biol 9:1255-1258.
48. Pajic, A., Spitkovsky, D., Christoph, B., Kempkes, B., Schuhmacher, M., Staege, M. S., Brielmeier, M., Ellwart, J., Kohlhuber, F., Bornkamm, G. W., et al. 2000. Cell cycle activation by c-myc in a burkitt lymphoma model cell line. Int J Cancer 87:787-793.
49. Athenstaedt, K., Weys, S., Paltauf, F., and Daum, G. 1999. Redundant systems of phosphatidic acid biosynthesis via acylation of glycerol-3-phosphate or dihydroxyacetone phosphate in the yeast Saccharomyces cerevisiae. J Bacteriol 181:1458-1463.
50. Hill, E. E., Husbands, D. R., and Lands, W. E. 1968. The selective incorporation of 14C-glycerol into different species of phosphatidic acid, phosphatidylethanolamine, and phosphatidylcholine. J Biol Chem 243:4440-4451.
51. Yang, Y., Cao, J., and Shi, Y. 2004. Identification and characterization of a gene encoding human LPGAT1, an endoplasmic reticulum-associated lysophosphatidylglycerol acyltransferase. J Biol Chem 279:55866-55874.
52. Nie, J., Hao, X., Chen, D., Han, X., Chang, Z., and Shi, Y. 2010. A novel function of the human CLS1 in phosphatidylglycerol synthesis and remodeling. Biochim Biophys Acta 1801:438-445.
53. Moon, Y. A., Ochoa, C. R., Mitsche, M. A., Hammer, R. E., and Horton, J. D. 2014. Deletion of ELOVL6 blocks the synthesis of oleic acid but does not prevent the development of fatty liver or insulin resistance. J Lipid Res 55:2597-2605.
54. Pauter, A. M., Olsson, P., Asadi, A., Herslof, B., Csikasz, R. I., Zadravec, D., and Jacobsson, A. 2014. Elovl2 ablation demonstrates that systemic DHA is endogenously produced and is essential for lipid homeostasis in mice. J Lipid Res 55:718-728.
55. Ofman, R., Dijkstra, I. M., van Roermund, C. W., Burger, N., Turkenburg, M., van Cruchten, A., van Engen, C. E., Wanders, R. J., and Kemp, S. 2010. The role of ELOVL1 in very long-chain fatty acid homeostasis and X-linked adrenoleukodystrophy. EMBO Mol Med 2:90-97.
56. Thupari, J. N., Pinn, M. L., and Kuhajda, F. P. 2001. Fatty acid synthase inhibition in human breast cancer cells leads to malonyl-CoA-induced inhibition of fatty acid oxidation and cytotoxicity. Biochem Biophys Res Commun 285:217-223.
57. Shiragami, R., Murata, S., Kosugi, C., Tezuka, T., Yamazaki, M., Hirano, A., Yoshimura, Y., Suzuki, M., Shuto, K., and Koda, K. 2013. Enhanced antitumor activity of cerulenin combined with oxaliplatin in human colon cancer cells. Int J Oncol 43:431-438.
58. Dang, C. V. 2013. MYC, metabolism, cell growth, and tumorigenesis. Cold Spring Harb Perspect Med 3.
59. Li, F., Wang, Y., Zeller, K. I., Potter, J. J., Wonsey, D. R., O'Donnell, K. A., Kim, J. W., Yustein, J. T., Lee, L. A., and Dang, C. V. 2005. Myc stimulates nuclearly encoded mitochondrial genes and mitochondrial biogenesis. Mol Cell Biol 25:6225-6234.
60. Lin, C. Y., Loven, J., Rahl, P. B., Paranal, R. M., Burge, C. B., Bradner, J. E., Lee, T. I., and Young, R. A. 2012. Transcriptional Amplification in Tumor Cells with Elevated c-Myc. Cell 151:56-67.
61. Shimizu-Albergine, M., Van Yserloo, B., Golkowski, M. G., Ong, S. E., Beavo, J. A., and Bornfeldt, K. E. 2016. SCAP/SREBP pathway is required for the full steroidogenic response to cyclic AMP. Proc Nati Acad Sci USA 113:E5685-5693.
62. McPherson, R., and Gauthier, A. 2004. Molecular regulation of SREBP function: the Insig-SCAP connection and isoform-specific modulation of lipid synthesis. Biochem Cell Biol 82:201-211.
63. Collier, J. J., Zhang, P., Pedersen, K. B., Burke, S. J., Haycock, J. W., and Scott, D. K. 2007. c-Myc and ChREBP regulate glucose-mediated expression of the L-type pyruvate kinase gene in INS-1-derived 832/13 cells. Am J Physiol Endocrinol Metab 293:E48-56.
64. Zhang, P., Metukuri, M. R., Bindom, S. M., Prochownik, E. V., O'Doherty, R. M., and Scott, D. K. 2010. c-Myc is required for the CHREBP-dependent activation of glucose-responsive genes. Mol Endocrinol 24:1274-1286.
65. Morita, S. Y., and Terada, T. 2015. Enzymatic measurement of phosphatidylglycerol and cardiolipin in cultured cells and mitochondria. Sci Rep 5:11737.
66. Houtkooper, R. H., and Vaz, F. M. 2008. Cardiolipin, the heart of mitochondrial metabolism. Cell Mol Life Sci 65:2493-2506.
67. Dolinsky, V. W., Cole, L. K., Sparagna, G. C., and Hatch, G. M. 2016. Cardiac mitochondrial energy metabolism in heart failure: Role of cardiolipin and sirtuins. Biochim Biophys Acta 1860:1544-1554.
68. Kiebish, M. A., Han, X., Cheng, H., Chuang, J. H., and Seyfried, T. N. 2008. Cardiolipin and electron transport chain abnormalities in mouse brain tumor mitochondria: lipidomic evidence supporting the Warburg theory of cancer. J Lipid Res 49:2545-2556.
69. Murray, N. R., and Fields, A. P. 1998. Phosphatidylglycerol is a physiologic activator of nuclear protein kinase C. J Biol Chem 273:11514-11520.
70. Griese, M., Gobran, L. I., and Rooney, S. A. 1991. ATP-stimulated inositol phospholipid metabolism and surfactant secretion in rat type II pneumocytes. Am J Physiol 260:L586-593.
71. Fickes, R., Voelker, D. R., Berry, K. Z., and Murphy, R. C. 2016. Tandem mass spectrometry of novel ether-linked phospholipid analogs of anionic pulmonary surfactant phospholipids. Rapid Commun Mass Spectrom 30:2601-2606.
72. Agassandian, M., and Mallampalli, R. K. 2013. Surfactant phospholipid metabolism. Biochim Biophys Acta 1831:612-625.
73. Svensson, R. U., Parker, S. J., Eichner, L. J., Kolar, M. J., Wallace, M., Brun, S. N., Lombardo, P. S., Van Nostrand, J. L., Hutchins, A., Vera, L., et al. 2016. Inhibition of acetyl-CoA carboxylase suppresses fatty acid synthesis and tumor growth of non-small-cell lung cancer in preclinical models. Nat Med 22:1108-1119.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of treating a subject for a MYC-driven neoplasm, the method comprising administering to the subject an effective amount of a lipogenesis inhibitor to treat the subject for the MYC-driven neoplasm.

2. The method according to claim 1, wherein the MYC-driven neoplasm is a MYC-driven renal cell carcinoma (RCC).

3. The method according to claim 1 or 2, wherein the method further comprises identifying the subject as having a MYC-driven neoplasm.

4. The method according to any of the preceding claims, wherein the administering results in regression of the MYC-driven neoplasm.

5. The method according to any of the preceding claims, wherein the lipogenesis inhibitor is an inhibitor of ACLY.

6. The method according to claim 5, wherein the inhibitor of ACLY is an anti-ACLY antibody, an ACLY inhibitory nucleic acid or a small molecule ACLY antagonist.

7. The method according to claim 6, wherein the small molecule ACLY antagonist is selected from the group consisting of: 3,5-Dichloro-2-hydroxy-N-(4-methoxy[1,1′-biphenyl]-3-yl)-benzenesulfonamide (BMS 303141); 3,3,14,14-Tetramethylhexadecanedioic acid (MEDICA 16); (3R,5S)-rel-5-[6-(2,4-Dichlorophenyl)hexyl]tetrahydro-3-hydroxy-2-oxo-3-furanacetic acid (SB 204990) and 8-Hydroxy-2,2,14,14-tetramethylpentadecanedioic acid (ETC-1002).

8. The method according to any of the preceding claims, wherein the lipogenesis inhibitor is an inhibitor of ACACA.

9. The method according to claim 8, wherein the inhibitor of ACACA is an anti-ACACA antibody, an ACACA inhibitory nucleic acid or a small molecule ACACA antagonist.

10. The method according to claim 9, wherein the small molecule ACACA antagonist is selected from the group consisting of: 5-(tetradecyloxy)-2-furancarboxylic acid (TOFA); [(3R)-1′-(9-anthracenylcarbonyl)[1,4′-bipiperidin]-3-yl]-4-morpholinyl-methanone (CP 640186); 1,4-Dihydro-1′-[2-methyl-1H-benzimidazol-6-yl)carbonyl]-1-(1-methylethyl)-spiro[5H-indazole-5,4′-piperidin]-7(6H)-one (PF 05175157); 2′-(tert-Butyl)-1-(2-methoxyquinoline-7-carbonyl)-4′,6′-dihydrospiro[piperidine-4,5′-pyrazolo[3,4-c]pyridin]-7′(2′H)-one; 1,17-Dihydroxy-10,11,18-trimethoxy-2,14,16-trimethyl-5-phenyl-4,19-dioxabicyclo[13.3.1]nonadec-12-en-3-one (Soraphen A); ND-630; ND-654; (S)-(+)-4-[1-(4-tert-Butylphenyl)-2-oxo-pyrrolidin-4-yl]methoxybenzoic acid (S-2E) and 8-hydroxy-2,2,14,14-tetramethylpentadecanediotic acid (ESP-55016).

11. The method according to any of the preceding claims, wherein the lipogenesis inhibitor is an inhibitor of FASN.

12. The method according to claim 11, wherein the inhibitor of FASN is an anti-FASN antibody, a FASN inhibitory nucleic acid or a small molecule FASN antagonist.

13. The method according to claim 12, wherein the small molecule FASN antagonist is selected from the group consisting of: Cerulenin; N-Formyl-L-leucine (1S)-1-[[(2S,3S)-3-hexyl-4-oxo-2-oxetanyl]methyl]dodecyl ester (Orlistat); (2R*,3S*)-Tetrahydro-4-methylene-2-octyl-5-oxo-3-furancarboxylic acid (C-75); 3-(3,4,5-Trihydroxybenzoyloxy)naphthalen-1-yl 3,4,5-trihydroxybenzoate (G-28UCM) and 4-[4-(5-Benzofuranyl)phenyl]-5-[[(3S)-1-(cyclopropylcarbonyl)-3-pyrrolidinyl]methyl]-2,4-dihydro-3H-1,2,4-triazol-3-one (GSK 2194069).

14. The method according to any of the preceding claims, wherein the lipogenesis inhibitor is an inhibitor of SCD.

15. The method according to claim 14, wherein the inhibitor of SCD is an anti-SCD antibody, a SCD inhibitory nucleic acid or a small molecule SCD antagonist.

16. The method according to claim 15, wherein the small molecule SCD antagonist is selected from the group consisting of: 4-(2-Chlorophenoxy)-N-[3-[(methylamino)carbonyl]phenyl]-1-piperidinecarboxamide (A 939572); 4-Pyridinecarboxylic acid 2-phenylhydrazide (PluriSln 1); 2-[5-[3-[4-(2-bromo-5-fluorophenoxy)piperidin-1-yl]-1,2-oxazol-5-yl]tetrazol-2-yl]acetic acid (MK-8245); 2-methyl-5-(6-(4-(2-(trifluoromethyl)phenoxy)piperidin-1-yl)pyridazin-3-yl)-1,3,4-thiadiazole (MF-438); 3-[4-(2-Chloro-5-fluorophenoxy)-1-piperidinyl]-6-(5-methyl-1,3,4-oxadiazol-2-yl)pyridazine (CAY 10566); N-(2-(6-(3,4-dichlorobenzylamino)-2-(4-methoxyphenyl)-3-oxopyrido[2,3-b]pyrazin-4(3H)-yl)ethyl) acetamide (CVT-11127); MF-152; LCF369; CVT-11,563; CVT-12,012; DSR-4029; GSK993 and HYR-061.

17. The method according to any of the preceding claims, wherein the method comprises administering to the subject an effective amount of two or more lipogenesis inhibitors.

18. A method of treating a subject for a lipogenesis-dependent neoplasm, the method comprising:
comparing a lipogenesis profile obtained from a subject having a neoplasm with a reference lipogenesis profile to classify whether the neoplasm is lipogenesis-dependent; and
administering to the subject an effective amount of a lipogenesis inhibitor, when the neoplasm is classified as lipogenesis-dependent, to treat the subject for the lipogenesis-dependent neoplasm.

19. The method according to claim 18, wherein the neoplasm is a renal cell carcinoma (RCC).

20. The method according to claim 18 or 19, wherein the neoplasm is classified as lipogenesis-dependent when the lipogenesis profile obtained from the subject comprises increased glycerophosphoglycerols compared to the reference lipogenesis profile.

21. The method according to any of claims 18 to 20, wherein the neoplasm is classified as lipogenesis-dependent when the lipogenesis profile obtained from the subject comprises decreased glycerophosphoinositols compared to the reference lipogenesis profile.

22. The method according to any of claims 17 to 19, wherein the lipogenesis profile obtained from the subject is a mass spectrometry (MS) lipogenesis profile.

23. The method according to claim 20, wherein the MS lipogenesis profile is a desorption electrospray ionization mass spectrometry imaging (DESI-MSI) lipogenesis profile.

24. A method of identifying a MYC-driven neoplasm therapeutic agent, the method comprising:
contacting a MYC-driven neoplasm with a candidate agent;
obtaining a candidate lipogenesis profile for the MYC-driven neoplasm following the contacting;
identifying the candidate agent as a MYC-driven neoplasm therapeutic agent when the candidate lipogenesis profile indicates decreased lipogenesis as compared to a control lipogenesis profile.

25. The method according to claim 24, wherein the MYC-driven neoplasm is a MYC-driven renal cell carcinoma (RCC).

26. The method according to claim 24 or 25, wherein the contacting is performed in vitro.

27. The method according to claim 24 or 25, wherein the contacting is performed in vivo.

28. The method according to claim 27, wherein the MYC-driven neoplasm is a human MYC-driven neoplasm xenograft.

29. The method according to any of claims 24 to 28, wherein the lipogenesis profile obtained is a mass spectrometry (MS) lipogenesis profile.

30. The method according to claim 29, wherein the MS lipogenesis profile is a desorption electrospray ionization mass spectrometry imaging (DESI-MSI) lipogenesis profile.

31. The method according to any of claims 24 to 30, wherein the control lipogenesis profile is the lipogenesis profile of the MYC-driven neoplasm prior to the contacting.

32. The method according to any of claims 24 to 30, wherein the control lipogenesis profile is a lipogenesis profile of noncancerous cells.

33. The method according to any of claims 24 to 30, wherein the control lipogenesis profile is a lipogenesis profile of a lipogenesis independent neoplasm.

34. The method according to any of claims 24 to 30, wherein the control lipogenesis profile is a lipogenesis profile of a non-MYC driven neoplasm.

35. The method according to any of claims 24 to 34, wherein the method further comprises measuring an activity of one or more lipogenesis genes or a protein expressed therefrom in the MYC-driven neoplasm following the contacting.

36. The method according to claim 35, wherein the one or more lipogenesis genes are selected from the group consisting of ACLY, ACACA, FASN and SCD.

37. The method according to any of claims 24 to 36, wherein the method further comprises identifying the candidate agent as a MYC-driven neoplasm therapeutic agent when the MYC-driven neoplasm regresses following the contacting.

38. The method according to any of claims 24 to 37, wherein the decreased lipogenesis comprises a decrease in glycerophosphoglycerols.

39. The method according to any of claims 24 to 38, wherein the candidate lipogenesis profile further indicates an increase in glycerophosphoinositols as compared to a control lipogenesis profile.

40. A method of treating a subject for a MYC-driven neoplasm, the method comprising administering to the subject an effective amount of a MYC-driven neoplasm therapeutic agent identified according to claims 24 to 39.
